(19) United States
(12) Patent Application Publication
(10) Pub. No.: US 2010/0023101 A1
(43) Pub. Date: Jan. 28, 2010

(54) BIOCOMPATIBLE COMPOSITES

(75) Inventors: Gordon George Wallace, Gwynnville (AU); Simon Edward Moulton, Figtree (AU); Philip Gregory Whitten, Fairy Meadow (AU); Carol Mary Lynam, North Wollongong (AU)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: University of Wollongong, North Wollongong (AU)

(21) Appl. No.: 12/278,233

(22) PCT Filed: Feb. 2, 2005
(86) PCT No.: PCT/AU2007/000104

§ 371 Date: Sep. 25, 2009

(30) Foreign Application Priority Data
Feb. 3, 2006 (AU) 2006900540

(51) Int. Cl. A61N 1/05; A61K 38/00; A61K 31/7088; A61F 2/00; A61B 5/0408

(52) U.S. Cl. 607/116; 514/2; 514/44 R; 424/423; 600/372; 977/742

(57) ABSTRACT

The present invention relates to biocompatible composites, in particular biocompatible nanotube composites in the form of a fiber mat and/or film structure, comprising nanotubes and at least one biomolecule. The invention also relates to a process for preparing a biocompatible composite involving (i) forming a dispersing media comprising nanotubes and at least one biomolecule; and either (ii) introducing the dispersing media of step (i) into a coagulating media optionally comprising at least one biomolecule so as to form a continuous fiber; or (iii) filtering the dispersing media of step (i). Alternatively, the process involves (i) forming a dispersing media comprising nanotubes; and (ii) introducing the dispersing media of step (i) into a coagulating media comprising at least one biomolecule so as to form a continuous fiber. The biocompatible composite is useful as a medical device, preferably in a bio-electrode, bio-fuel cell or substrates for electronically stimulated bio-growth.

### FIELD

The present invention relates to biocompatible composites, in particular biocompatible nanotube composites for use in medical applications requiring electrical conduction or sensing such as bio-electrodes.

### BACKGROUND

Bio-electrodes are used to deliver charge to, or sense electric pulses on or within living organisms. Common bio-electrodes include pacemaker electrodes and electrocardiogram (ECG) pads. A pacemaker electrode needs a high capacitance to overcome the pacing threshold, while exhibiting a low polarization so that it can successfully detect cardiac signals.

The interaction between an electrode and a living organism is essential to its long term use. An electrode must be biocompatible, so that it is not toxic to the living organism in which it is implanted. Controlling the response of the body to an implanted electrode is also critical to its long-term use. For pacemaker electrodes, many materials are biocompatible, but the body responds by enveloping them in fibrous tissue which increases the threshold charge for stimulation. There is still much potential to improve pacemaker electrodes by increasing their surface area, and decreasing the amount of fibrous tissue that envelopes them when they are implanted.

Commercial implantable bio-electrodes for humans are made from Pt and Pt—Ir alloys. Often these metals are coated with TiN or conducting oxides (eg. RuO₂ or IrO₂) to increase their surface area, or adjust their bio-interaction.

Carbon nanotubes (CNTs) represent a new material from which to construct macroscopic electrodes. Assemblies of CNTs without a binder (e.g. bucky paper) and with a binder have been promoted for several electrode applications including super capacitors and batteries. These applications exploit the large surface area and the low chemical reactivity of the CNTs. Compared to conventional metallic electrodes, CNT assemblies exhibit an order of magnitude decrease in conductivity, but a similar increase in surface area.

The ability to process CNTs provides potential advantages of these materials in a variety of applications. Due to their chemical inertness and strong inner-tube van der Waals attractions, CNTs aggregate into ropes with limited solubility in aqueous, organic, or acidic media. Because of the high temperature stability of CNTs, melt spinning is not an option. Particle coagulation spinning, in the case for rod like polymers, is an attractive processing approach which produces CNT fibres. The main challenge to the production of CNT fibres is dispersing nanotubes at high enough concentrations suitable for efficient alignment and effective coagulation.

CNTs have been assembled into long ribbons and fibres by dispersing them in an aqueous surfactant solution and then re-condensing the dispersion in a stream of a synthetic polymer solution (polyvinyl alcohol) to form a fibre. However if a surfactant is used to disperse CNTs, there is the added complication of removing the surfactant from the fibre during coagulation or after processing.

Fibres currently available vary in terms of internal structure, density and purity and their mechanical properties reported to date are only a fraction of those obtained for individual nanotubes. Electronic conductivity measurements of fibres with polymer and/or dispersant present are also very low in comparison to individual nanotubes.

There is a need for nanotube composites which are biocompatible, electrically conducting and robust.

### SUMMARY

The present invention provides a biocompatible composite that is formed into a fibre, mat and/or film structure, comprising nanotubes and at least one biomolecule.

The composite can be prepared by using the biomolecule as a dispersant and/or coagulant.

The present invention also provides a process for preparing a biocompatible composite which comprises the steps of:
(i) forming a dispersing media comprising nanotubes and at least one biomolecule; and either
(ii) introducing the dispersing media of step (i) into a coagulating media optionally comprising at least one biomolecule so as to form a continuous fibre; or
(iii) filtering the dispersing media of step (i).

Preferably at least one biomolecule is present in both the dispersing media of step (i) and the coagulating media of step (ii). It has also been found effective for ionic biomolecule coagulants to possess an opposite charge to ionic biomolecule disperants.

The present invention further provides a process for the preparation of a biocompatible composite which comprises the steps of:
(i) forming a dispersing media comprising nanotubes; and
(ii) introducing the dispersing media of step (i) into a coagulating media comprising at least one biomolecule so as to form a continuous fibre.

In one embodiment, the dispersing media forms a continuous fibre by being spun into the coagulating media. As the biocompatible composites of the present invention are highly conductive and robust they may be woven into mats or yarns or knitted into structures for medical applications, requiring electrical conduction or sensing such as bio-electrodes for example pacemaker electrodes and ECG pads. Alternatively, the filtered mats may be used in that form.

Thus, the present invention further provides a medical device such as a bio-electrode which is composed wholly or partly of the biocompatible composite defined above.

### DETAILED DESCRIPTION

### Structure of Composite

Nanotubes are typically small cylinders made of organic or inorganic materials. Known types of nanotubes include CNTs, metal oxide nanotubes such as titanium dioxide nanotubes and peptidyl nanotubes. Preferably the nanotubes are CNTs.

CNTs are sheets of graphite that have been rolled up into cylindrical tubes. The basic repeating unit of the graphite sheet consists of hexagonal rings of carbon atoms, with a carbon-carbon bond length of about 1.45 Å. Depending on how they are made, the nanotubes may be single-walled nanotubes (SWNTs) or multi-walled nanotubes(MWNTs). A typical SWNT has a diameter of about 1.2 to 1.4 nm.

The structural characteristics of nanotubes provide them with unique physical properties.

Nanotubes may have up to 100 times the mechanical strength of steel and can be up to 2 mm in length. They exhibit the electrical characteristics of either metals or semiconductors, depending on the degree of chirality or twist of the nanotube. Different chiral forms of nanotubes are known as armchair, zigzag and chiral nanotubes. The electronic properties of carbon nanotubes are determined in part by the diameter and length of the tube.

The term “biomolecule” generally refers to molecules or polymers of the type found within living organisms or cells and chemical compounds interacting with such molecules. Examples include biological polyelectrolytes such as hyaluronic acid (HA), chitosan, heparin, chondroitin sulphate, polyglycolic acid (PGA), polylactic acid (PLA), polyamides, poly-2-hydroxy-butyrate (PHB), polycaprolactone (PCL), poly(lactic-co-glycolic)acid (PLGA), protamine sulfate, polyallylamine, polydiallyldimethylammonium, polyethyleneimine (PEI), eudragit, gelatin, spermidine, albumin, polyacrylic acid, sodium alginate, polystyrene sulfonate, carrageenin, carboxymethylcellulose; nucleic acids such as DNA, cDNA, RNA, oligonucleotide, oligoribonucleotide, modified oligonucleotide, modified oligoribonucleotide and peptide nucleic acid (PNA) or hybrid molecules thereof; polyaminoacids such as poly-L-lysine, poly-L-arginine, poly-L-aspartic acid, poly-D-glutamaic acid, poly-L-glutamaic acid, poly-L-histidine and poly-(DL)-lactide; proteins such as growth factor receptors, catecholamine receptors, amino acid derivative receptors, cytokine receptors, lectins, cytokines and transcription factors; enzymes such as proteases, kinases, phosphatases, GTPases and hydrolases; polysaccharides such as cellulose, amylose and glycogen; lipids such as chylomicron and glycolipid; and hormones such as amino-derived hormones, peptide hormones and steroid hormones. Preferred examples include hyaluronic acid, chitosan, heparin, chondroitin sulphate, DNA, polyethyleneimine and/or poly-L-lysine.

Polyelectrolytes are polymers having ionically dissociable groups, which can be a component or substituent of the polymer chain. Usually, the number of these ionically dissociable groups in the polyelectrolytes is so large that the polymers in dissociated form (also called polyions) are water-soluble. Depending on the type of dissociable groups, polyelectrolytes are typically classified as polyacids and polybases. When dissociated, polyacids form polyanions, with protons being split off, which can be inorganic, organic and biopolymers. Polybases contain groups which are capable of accepting protons, e.g., by reaction with acids, with a salt being formed.

The structures of some biomolecules suitable for use in the composite of the present invention are set out below:

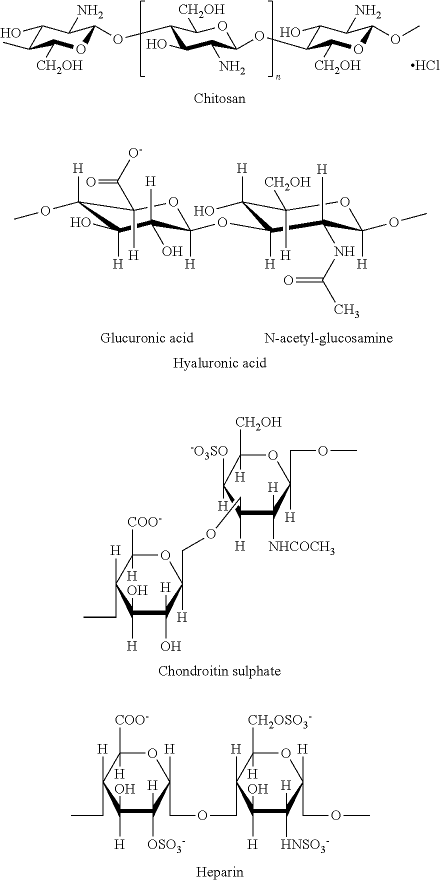

It will be appreciated that the biomolecule may include functional groups to allow further control of the biointeraction such as biomolecules which convey active ingredients for example drugs, hormones, growth factors or antibiotics. The biomolecule can also be chosen depending on the desired application, for example, if the composite was to be used to promote or inhibit adhesion of certain cell types it may be advantageous to use biomolecules which promote nerve or endothelial cell growth or inhibit smooth muscle cell growth (fibroblasts).

More than one biomolecule may be present in the composite. In one embodiment, there are biomolecules present in both the dispersing and coagulating media. It has been found effective for ionic biomolecule coagulants to possess a charge opposite to the ionic biomolecules of the dispersion. For example, the sodium salts of DNA and HA were used as dispersants, creating suspensions of biomolecules with a net negative charge and it was found that biomolecules with a positive charge, e.g. chitosan hydrochloride, were effective as coagulants. Similarly, chitosan hydrochloride as a dispersant is effectively coagulated by biopolymers with a net negative charge, e.g. HA, chondroitin sulphate sodium salt and heparin sodium salt. This suggests that composite formation is governed by charge neutralisation and re-saturation.

Examples of suitable composites of the present invention include:

DNA-SWNT-chitosan fibres;

HA-SWNT-chitosan fibres;

HA-SWNT-PEI fibres;

Chitosan-SWNT-chondroiton sulphate fibres;

Chitosan-SWNT-heparin fibres;

Chitosan-SWNT films;

Chitosan-SWNT-PEI fibres

DNA-SWNT films; and

Poly-1-lysine-SWNT films.

The biomolecule may be present in an amount in the range of 10-50% based on the total weight of the composite.

The composite may include other biocompatible additives depending on the desired application including drugs, growth factors, hormones, antibiotics, mRNA, DNA, steroids, antibodies and radio-isotopes which could be incorporated into the biomolecule or added to the dispersing and/or coagulating media during preparation of the composite.

The additive may be present in an amount in the range of 1-50% based on the total weight of the composite.

While the composite is in the form of fibres, films or mats, these could be of any dimension including three dimensional structures such as hollow fibres which could be achieved by filtering the composite through a tube.

### Preparation of the Composite

The preparation of the composite involves a first step of forming a dispersing media containing the nanotubes. The biomolecule is usually introduced into the dispersing media at this stage, although it may be introduced in a second step as part of the coagulating media.

The term “media” is used in its broadest sense and refers to any media which is capable of dispersing and/or coagulating the nanotubes and the biomolecules if present.

While the media is generally a solution it may have a viscosity of up to about 200 cp. The solution usually contains a solvent such as water, acetic acid, toluene, ethanol or methanol which will be chosen depends on the type of nanotubes and biomolecules employed. The dispersing media may be heated prior to the dispersion step.

Dispersion generally involves sonication which may be performed using any suitable known technique such as immersing a sonicator such as an ultrasonic horn into the dispersing media containing the nanotubes, solvent and biomolecule if present.

The ratio of biomolecule to nanotubes may be in the range of 1:1 to 5:1.

The concentration of nanotubes in the dispersing media is generally in the range of 0.2 to 0.5 wt %.

The dispersion step forms stable biomolecule-nanotube suspensions which may then be subjected to either coagulation or filtering.

The coagulation step is performed to produce continuous fibres which may range in length from centimetres to metres depending on the desired application.

Coagulation involves spinning the nanotube or biomolecule-nanotube dispersion into a coagulating media. The coagulating media may contain the same or a different biomolecule to that used in the dispersing media, no biomolecule when a biomolecule has been used in the dispersing media or the only biomolecule present in the composite when the dispersing media just contains nanotubes. When the composite contains two or more different biomolecules, it has been found it is advantageous to composite formation for an ionic biomolecule coagulant to possess a charge opposite to that of an ionic biomolecule dispersant.

The dispersion may be spun into the coagulating media using any suitable known technique including injecting the dispersion through an orifice such as a needle into the spinning coagulating media. The injection rate and spinning speeds are adjusted depending on the composite being formed. Typical injection rates are in the range of 150 to 300 ml/hr and spinning speeds are in the range of 25 to 60 rpm. The fibres may have diameters in the range of 20 to 200 μm and may also be in the form of ribbons having a thickness in the range of 15 to 50 μm. Hollow fibres can also be formed using this technique by varying the composition of the coagulating media.

Alternatively, the dispersion containing the biomolecule is not subjected to coagulation and just filtered over a porous polymer filter membrane or other porous material after step (i) using any suitable known technique including vacuum filtration and pressure filtration so as to form films or mats being in the range of 50 to 100 μm in thickness. The filtering step can also be used to produce three dimensional shapes including hollow fibres by filtering through a tube.

The composite may be washed for example in deionised water and/or dried at ambient temperatures or under vacuum after the coagulation or filtering steps.

### Properties of Composite

The composite of the present invention is biocompatible, mechanically robust and has electrical conductivities which make it suitable for use as a bio-electrode.

Biocompatibility studies were performed by screening the growth of L-929 cell culture on chitosan-SWNT and DNA-SWNT composites and prolific cell growth was observed.

The composite of the present invention possesses the following physical properties:

Tensile Stress (MPa): 50-200 MPa

Elastic Modulus (GPa): 1-20 GPa

Density (kg/m³): 0.6-1 g/m³

The incorporation of the biomolecule in the composite results in a substantial increase in mechanical strength. The modulus may also be increased. Preventing nanotube junctions from slipping by the adhesion of a biomolecule is also a possible strengthening mechanism.

For many brittle materials or composites the observed strength is determined by the size and density of defects (eg. small cracks). For example, glass fibre exhibits a much higher tensile strength than glass sheet. For bucky paper, a major defect with respect to stress concentration would be large pores and the connection between bundles of nanotubes. It is feasible that the biomolecule would substantially increase the strength by filling in some of the pores, and thereby increasing the strength between clumps.

The specific strength of the composites is at the upper limits of steel or aluminium alloys, but at the lower limit for commercial glass fibre reinforced polymers. The tensile strength of the chitosan-SWNT is better than most common engineering polymers, with only oriented fibres (eg. Nylon or polyethylene) being stronger.

An interesting feature of the HA-SWNT-Chitosan fibre is revealed when tying knots, in that the fibre does not break as the knot is tightened. This implies that the fibre can be curved through 360° in a few micrometres, which demonstrates a robust nature, flexibility of the fibre and a high resistance to bending when compared to classical carbon fibres.

The electrical conductivity of the composite is in the range of 0.5-400 S/cm.

Composites of DNA-SWNT and chitosan-SWNT exhibited significantly higher conductivity than that of standard bucky paper. It is surprising that the addition of a non-conductive biomolecule results in an increase of conductivity. Most composites composed of non-conductive binders and carbon nanotubes report conductivities less than 10 S/cm. It was expected that the composite conductivity should be lower on the basis that the non-conductive binder insulates the nanotubes from themselves, and hence limits the number of conductive pathways.

The observed increase in conductivity is most likely due to poor dispersion. Within the large agglomerates of nanotubes the local conductivity would be very high, with minimal biomolecule hindering nanotube-nanotube contact. However, electrical contact between bundles is limited by the presence of the insulative biomolecule. Hence, the intra-bundle conductivity would be relatively high, the inter-bundle conductivity relatively low, and the composites conductivity determined by the density of electrical contacts between bundles.

The electrical conductivity, of DNA-SWNT-PVA fibres has previously been reported as 0.04 S cm⁻¹, which is almost three orders of magnitude lower than the DNA-SWNT-chitosan composite of the present invention. Conductivities of up to 130 S cm-1 have been measured for the HA-SWNT-chitosan fibre of the present invention, which is four times higher than for as-produced carbon nanotube fibres reported previously. Carbon nanotube fibres, which have been wet-spun from polymer solutions, have until now, been reported with modest conductivities. Annealed fibres have been reported with conductivities as high as 167 S cm⁻¹. It is believe that this is the first report of as-spun fibres with high electrical conductivity.

### Potential Applications of Composite

As the composites of the present invention are biocompatible and electrically conductive they could be used in medical applications that require electro-stimulation, the passage of an electrical current or electrical sensing such as bio-electrodes, biofuel cells or as substrates for electrically stimulated bio-growth.

Bio-electrodes are one application of the composites of the present invention. The composites exhibit sufficient conductivity, electrochemical capacitance and mechanical properties to be used directly as electrodes implanted into living organisms for the purpose of electrical sensing and stimulation. Specific applications include pacemaker electrodes, ECG pads, biosensors, muscle stimulation, epilepsy control and electrical stimulated cell regrowth.

Electrodes for biological implants typically consist of platinum or iridium and their derivatives. The present invention provides electrically conducting composites that contain only biomolecules and nanotubes. Biomolecules such as chitosan are known to be biocompatible and are currently used in conjunction with many implants in the human body. Furthermore, functional groups may be added to chitosan to allow further control of the bio-interaction. The bio-compatibility of carbon nanotubes is not known, however initial studies show great promise. Therefore, potentially a new bio-electrode which is robust and efficient has been produced. These bio-electrodes should also be efficient and robust.

### DESCRIPTION OF THE DRAWINGS

In the examples which follow, reference will be made to the accompanying drawings in which:

FIG. 1 is a schematic diagram showing spinning CNT-bio-fibres and ribbons from CNT-biomolecule dispersions into a coagulation bath;

FIG. 2 shows high resolution SEM images of SWNT-Bio-Fibres, showing differences in fibre surface morphology (top left: DNA-SWNT-Chitosan, top right:

HA-SWNT-Chitosan, bottom left: Chitosan-SWNT-Chondroitin sulphate, bottom right: Chitosan SWNT-Heparin);

FIG. 3 shows high resolution SEM images of fractured ends of SWNT-Bio-Fibres showing SWNT bundles coated with biomolecules (top left: DNA-SWNT-Chitosan, top right: HA-SWNT-Chitosan, bottom left:

Chitosan-SWNT-Chondroitin sulphate, bottom right: Chitosan-SWNT-Heparin);

FIG. 4 shows Raman spectra of SWNT-Bio-Fibres confirming presence of SWNTs.

FIG. 5 shows a schematic diagram of the preparation of the biocompatible CNT film of Example 2;

FIG. 6 shows an optical microscope image of a DNA dispersion (Scale bar is 200 μm);

FIG. 7 shows an optical microscope image of a Chitosan dispersion (Scale bar is 200 μm);

FIG. 8 shows anoptical microscope image of Triton X 100 dispersion (Scale bar is 200 μm);

FIG. 9 is a photograph of the composite samples prepared in Example 2;

FIG. 10 is an SEM image of the filter surface of standard bucky paper. [SEM of Triton X nanotube surface];

FIG. 11 is an SEM image of the filter surface of the DNA-SWNT composite;

FIG. 12 is an SEM image of the filter surface of the chitosan-SWNT composite; and

FIG. 13 is a photograph showing L-929 cells growing on the DNA-SWNT composite.

### EXAMPLES

The invention will now be described with reference to the following non-limiting examples.

### Example 1

### Preparation of Biocompatible CNT Continuous Fibres

### Materials

A phosphate buffered saline solution (PBS—0.2M pH 7.4) was prepared as described. All other chemicals, Single Wall Carbon Nanotubes (SWNT, HiPCo produced from CNI), salmon sperm DNA (Nippon Chemical Feed Co. Ltd.—Japan), hyaluronic acid (Sigma), chitosan (Jakwang Co. Ltd.), chondroitin sulphate (ICN-Biochemicals—Ohio, USA), heparin (Sigma), potassium ferricyanide (Sigma) were used as received.

### Instrumentation

SEM images were acquired using a Hitachi S-900 field-emission scanning electron microscope (FESEM) Samples for FESEM were sputter coated with chromium prior to analysis. The nanotube films were imaged with no coating.

Raman spectroscopy measurements were performed using a Jobin Yvon Horiba HR800 Spectrometer equipped with a He:Ne laser operating at a laser excitation wavelength of 632.8 nm utilizing a 300-line grating.

Electrical conductivity measurements were carried out using a conventional four-point probe method at room temperature.

Electrochemical capacitance was calculated from the slope of anodic current amplitude when graphed against the scan rate, obtained from cyclic voltammetry at different potential scan rates, in phosphate buffered saline solution (PBS—0.2M pH 7.4) with Ag/AgAl reference electrode. Cyclic Voltammetry were performed using an eDAQ e-corder (401) and potentiostat/galvanostat (EA 160) with Chart v5.1.2/EChem v 2.0.2 software (ADInstruments) and a PC computer.

### Procedure

DNA-SWNT dispersions were prepared from an aqueous solution of DNA (0.4 wt %) containing SWNT in a ratio of 1:1, which was sonicated for 30 minutes using a high power sonic tip (500W) DNA-SWNT-chitosan composite fibres were prepared from a DNA-SWNT dispersion, 1:1 (0.4 wt %), utilising a rotating aqueous chitosan coagulant solution (0.2 wt %). Following coagulation, fibres were washed with de-ionised water prior to drying in ambient conditions.

HA-SWNT-chitosan fibres were spun from a HA-SWNT dispersion, 1:1(0.4 wt %), utilising an aqueous chitosan coagulant (0.2 wt %) in a manner described for the DNA-SWNT-chitosan fibres.

Chitosan-SWNT-chondroitin sulphate fibres were produced from a chitosan-SWNT dispersion, 2:1 (0.3 wt %) and chondroitin sulfate coagulant (0.5 wt %) in a similar manner to the DNA-SWNT-chitosan fibres.

Chitosan-SWNT-heparin fibres were spun from a chitosan-SWNT dispersion 2:1 (0.3 wt %), and a heparin coagulation solution (0.5 wt %) in a similar manner to the DNA-SWNT-chitosan fibres.

### Results

DNA, chitosan and HA are examples of biomolecules which effectively disperse SWNTS. These SWNT-biomolecule dispersions were obtained by sonicating a given amount of SWNTs in an aqueous solution of biomolecule, to form highly stable biomolecule-SWNT suspensions. In the case of DNA and HA as dispersant, concentrations of 0.4% by weight of SWNTs were used. To obtain a homogenous dispersion, a 1:1 ratio by weight of SWNT: biomolecule was necessary. This is in contrast with reports published using molecular surfactants where ratios of at least 2:1¹, and in some cases 3:1², were required. Actually the literature states for DNA that 1:1 or higher is sufficient.³ In the case of chitosan as dispersant, a concentration of 0.3% by weight of SWNTs was used; however it was necessary to employ a 2:1 ratio by weight of SWNT:chitosan to obtain a homogenous dispersion. Dispersions differing from this concentration and ratio contained large clusters, between 50 and 100 μm in size, of non-disperse SWNTs. High chitosan concentrations appeared to induce the formation of SWNT aggregates as in the case of molecular surfactants, which is in contrast to DNA and HA.

A coagulation drop test was performed to deduce the suitability of dispersion:coagulating-polymer combinations and the results can be found in Table 1 below. Preliminary results imply that effective ionic coagulants possess a charge opposite to that of the dispersion. Since the sodium salts of DNA and HA were used as dispersants, creating suspensions of biomolecules with net negative charge, it was found that biopolymers with a positive charge, e.g. chitosan hydrochloride, were effective as coagulants. Similarly, chitosan hydrochloride as dispersant is effectively coagulated by biopolymers with a net negative charge, e.g. HA, chondroitin sulphate sodium salt and heparin sodium salt. This suggests that fibre formation may be governed by charge neutralisation and re-saturation as is the case for lipid bilayer formation.

TABLE 1
Biomolecule-SNWT dispersions tested with biomolecules as coagulants

| | | Coagulant | |
|---|---|---|---|
| DNA | Heparin | Polylysine | Chitosan |
| | X | ✓ | ✓ |
| Hyaluronic Acid | Heparin | Polylysine | Chitosan |
| | X | ✓ | ✓ |
| Chitosan | Heparin | Hyaluronic Acid | Chondroitin Sulfate |
| | ✓ | ✓ | ✓ |

X: could not be pulled out of solution
✓: could be pulled out of solution

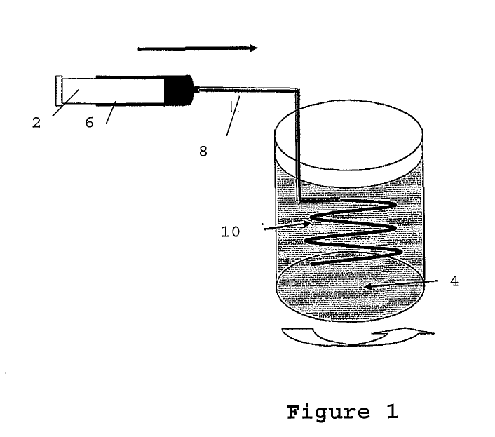

Employing injection rates and spinning speeds found to be most favourable, the SWNT-biomolecule dispersions 2 were injected using a syringe pump 6 via a needle and spun into a coagulation bath 4 to form CNT-bio-fibres and ribbons as shown in FIG. 1. The coagulation bath 4 consisted of appropriately charged aqueous soluble and biocompatible polymers, e.g. chitosan for DNA and HA dispersions and chondroitin sulphate and heparin for chitosan dispersions. In the case of the DNA-SWNT-chitosan and HA-SWNT-chitosan fibres, a wide variety of dispersion injection speeds were possible (150-300 ml/hr) along with coagulation rotation speeds between 25-60 rpm. DNA-chitosan fibres shrunk greatly upon drying to form approximately uniform cylindrical fibres. HA-chitosan fibres were ribbon-like in structure and possessed many kinks along the fibre due to the rotation of the coagulation bath. Chitosan-heparin sulfate fibres were produced, using a dispersion injection speed of 200 ml/hr with a coagulation rotation speed of 15 rpm. These fibres were generally uniform cylindrical fibres, with a corrugated surface. Chitosan-chondroitin sulfate fibres were produced, using a dispersion injection speed of 200 ml/hr with a coagulation rotation speed of 25 rpm. These fibres were ribbon-like in structure, possessing kinks along the fibre. Upon drying the ribbons curled to form more compact fibre structures.

Fibre lengths of up to one metre could be made using optimal conditions; however typical fibre lengths were 30cm to avoid entanglement in the rotating coagulation bath. Typical fibre diameters are as follows:

DNA-SWNT-chitosan fibre: 20-50 μm

Chitosan-SWNT-heparin fibre: 70-100 μm

In the case of the HA-SWNT-chitosan and chitosan-SWNT-chondroitin sulphate fibres, ribbons were formed in contrast to the cylindrical fibre morphology of the DNA-SWNT-chitosan and chitosan-SWNT-heparin fibre. Typical ribbon-like fibre widths (w) and thicknesses (t) are as follows:
HA-SWNT-chitosan fibre: 100-200 μm (w) 15-50 μm (t) Chitosan-SWNT-chondroitin
sulphate fibre : 100-120 μm (w) 30-40μm (t)

### Electrical Properties

The electrical conductivity, of DNA-SWNT-PVA fibres has previously been reported as 0.04 S cm⁻³, which is almost three orders of magnitude lower than the DNA-SWNT-chitosan fibre (see Table 2 below). Conductivities of up to 130 S cm⁻¹ have been measured for the HA-SWNT-chitosan fibre, which is four times higher than for as-produced carbon nanotube fibres reported previously^{1,2}. Carbon nanotube fibres, which have been wet-spun from polymer solutions, have until now, been reported with modest conductivities. Annealed fibres have been reported with conductivities as high as 167 5 cm^{-1 3}. It is believed that is the first report of as-spun fibres with high electrical conductivity.

TABLE 2
Conductivity measurements of bio-fibres

| | | Conductivity | |
|---|---|---|---|
| Dispersion | Coagulant | (S cm⁻¹) | St. Dev. (%) |
| DNA-SWNT | Chitosan | 29.5 | 14 |
| HA-SWNT | Chitosan | 134.6 | 35 |
| Chitosan- SWNT | Chondroitin | 1.5 | 2 |
| Chitosan- SWNT | Heparin | 0.4 | 12 |

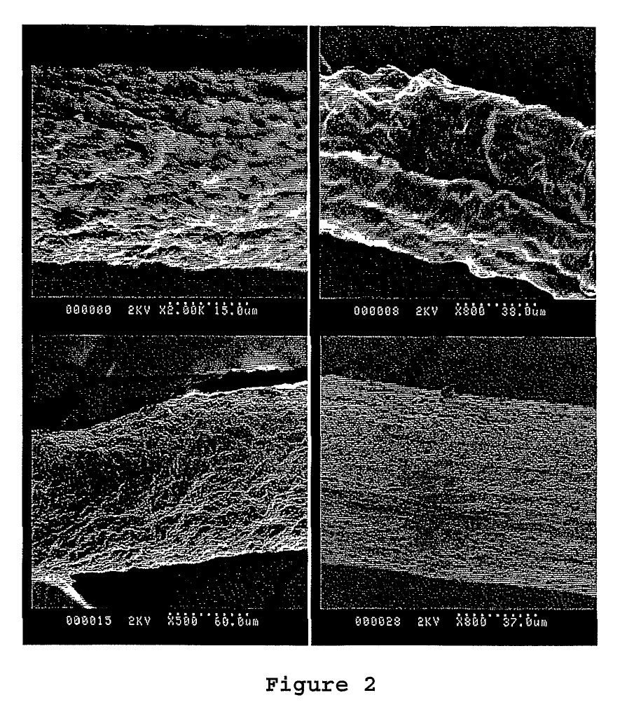

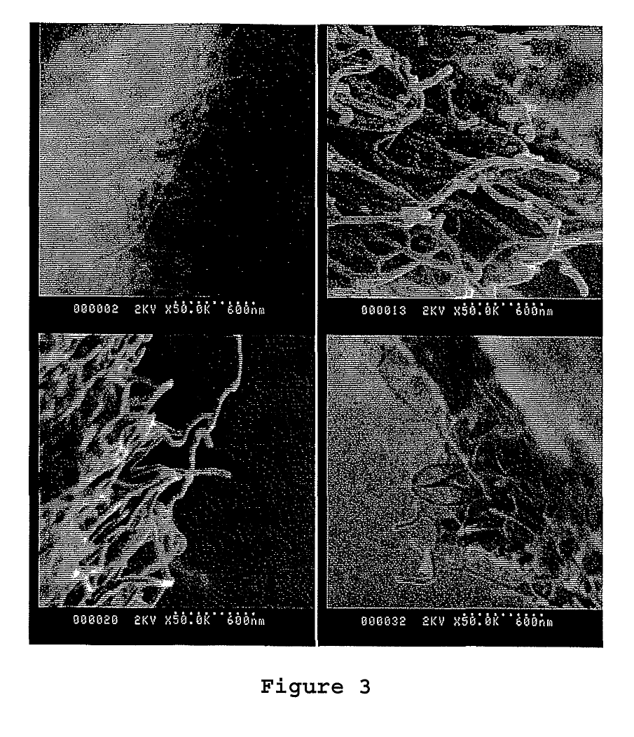

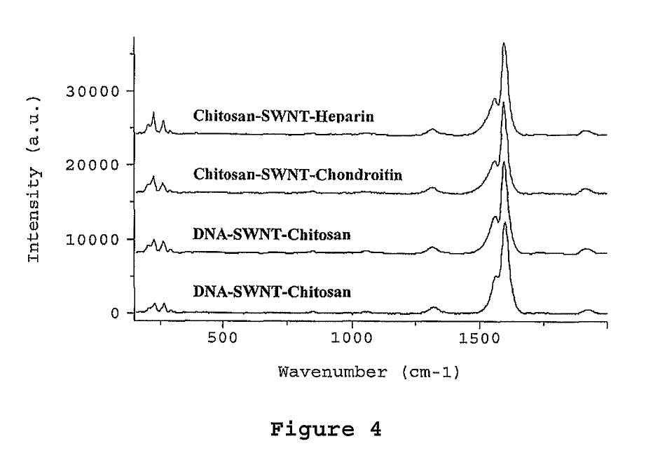

Raman spectroscopy and microscopy characterisation have confirmed the presence of CNTs in the fibres (see FIGS. 2 to 4).

### Electrochemical Properties

Electrochemical characterisation of these conducting bio-fibres was performed in phosphate buffered saline and buffered potassium ferricyanide.

### Mechanical Properties

An interesting feature of the HA-SWNT-chitosan fibre is revealed when tying knots, in that the fibre does not break as the knot is tightened. This implies that the fibre can be curved through 360° in a few micrometers, which demonstrates a robust nature, the flexibility of the fibre and high resistance to torsion when compared to classical carbon fibres.

Preliminary results indicate that the average tensile strength of the chitosan-chondroitin sulphate and chitosan-heparin fibres is 170 MPa and 118 MPa respectively. Young's modulus of the chitosan-chondroitin sulphate and chitosan-heparin fibres is 90 MPa and 80 MPa respectively.

### Example 2

### Preparation of Biocompatible CNT Films

### Materials and Procedure

SWNTs were obtained from CNI (batch P0276) and used without any further treatment. DNA (M_{w} 6.0×10⁶—lot no. 04056) purified from salmon sperm was obtained from Nippon Chemical Feed Co. Ltd., Japan. Chitosan (M_{w} of 2.0×10⁵) was obtained from Jakwang Co. Ltd., South Korea. Poly-L-lysine hydrochloride (Mw of 8.3×10⁴) was obtained from Aldrich. For dispersions, 40 mg of SWNT was combined with 40 mg of Chitosan, DNA or poly-L-lysine. For the DNA dispersions, 80 ml distilled water was added, and the solution heated to boiling prior to sonication. For the poly-L-lysine dispersions, 80 ml of distilled water was added. For the chitosan dispersions, 80 ml of 2-4 wt % acetic acid was added. Both chitosan and DNA solutions containing SWNT were dispersed for 1 hour with an ultrasonic horn (Sonics and Materials Inc. 500 Watt Vibra cell).

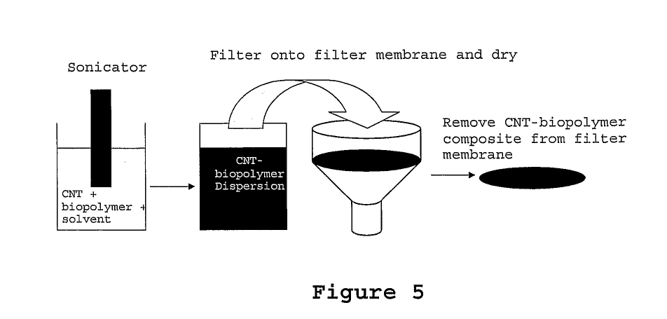

Following dispersion, the respective solutions were vacuum filtered over a 0.1-0.22 μm membrane forming films 20-100 μm in thickness. In the present method no washing of the sample is performed. Prior to testing, the respective films were dried under vacuum for 24 hours. A schematic diagram of the composite preparation shown in FIG. 5.

For comparison, a standard piece of bucky paper was made using the technique reported⁴. Briefly, 40 mg of the SWNT was dispersed using 1 wt % Triton X-100 surfactant (Aldrich) in 80 ml of water for 1 hour using an ultrasonic horn. The dispersions were then vacuum filtered, and washed with distilled water and methanol.

To evaluate the quality of dispersion following sonication, a small quantity of the dispersion was placed between two glass slides and imaged with a transmission optical microscope. Mechanical tensile testing was performed with an Instron universal testing machine, and TA instruments DMAQ800. Mechanical measurements were made on samples immediately taken from their vacuum storage, and samples immediately after they had been submerged in water for different periods of time. Conductivity measurements were performed with a 4 point conductivity probe. Electrochemical capacitance was performed using a Princeton Technology 363 potentiostat, in a 1 M NaNO₃ electrolyte with Ag|AgCl reference electrode. The current amplitude was measured at different potential scan rates (1-60 mV/s), with the capacitance being half of the slope of the current amplitude when graphed against the scan rate. Scanning electron microscope (SEM) images were obtained with a Leica Stereoscan 440 SEM.

Thermal gravimetric analysis (TGA) was performed with a TA instruments Q600. From TGA the weight percent binder and residual were calculated. The weight binder was taken to be the percent weight loss between 110 and 330° C. (assuming no weight loss at 110° C.). The residual is the percent weight remaining after heating to 700° C.

The percent of binder retained is the weight percent of total binder in filtration solution that has been retained in the SWNT film.

Each of the SWNT composites were screened for biocompatibility by the growth of L-929 cell culture. For the cell culture study, each sample was soaked overnight in culture media, then rinsed consecutively with water and a 70% ethanol:30% water mixture. The samples were then sterilized under a UV light for 20 minutes. Then the samples were placed into wells (96 well plate), with each well being seeded with 5000 cells and cultured for 72 hours, Finally the cells were stained with calcein and imaged. Please note that calcein fluoresces in metabolically active cells.

### Results

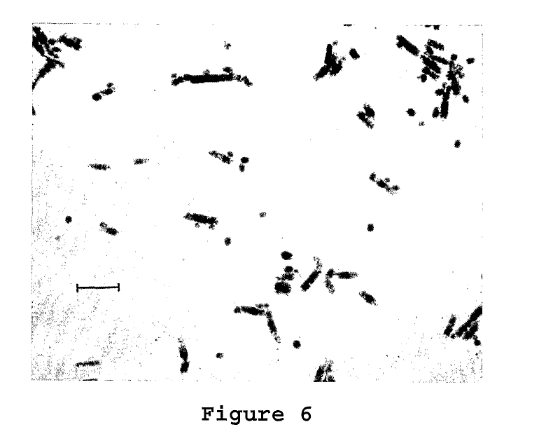

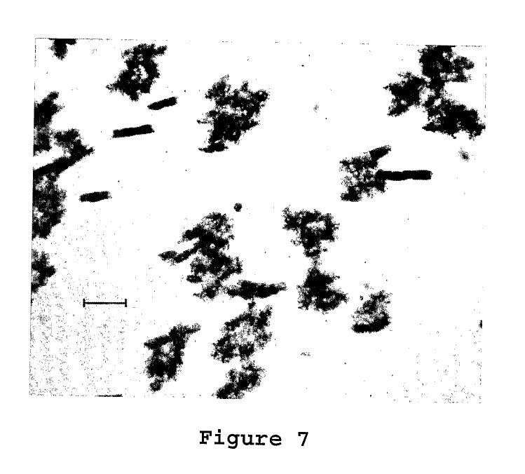

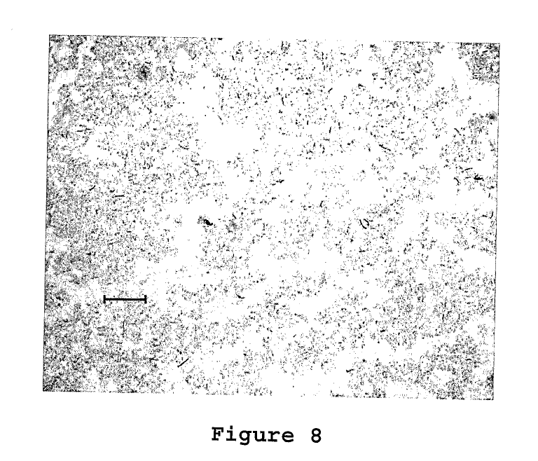

The degree of dispersion in Chitosan and DNA solutions was very coarse, with many nanotubes present as long rod like clumps with many exceeding 200 μm in length (see FIGS. 6 and 7). The variation between FIGS. 6 and 7 can be observed within one sample, and is not thought to be indicative of a difference in dispersions. In contrast, the dispersion created by triton X (see FIG. 8) was more fine, with many smaller clumps (≦50 μm in length) present than for the chitosan or DNA dispersions (see FIG. 7). The stability of the chitosan/DNA dispersions was much higher than for the triton X. If a chitosan/DNA dispersion was left for several days, there was only a minor settlement of black deposit on the bottom of the flask. In contrast, there was substantial settlement of a black deposit on the bottom of the flask for the triton X dispersions.

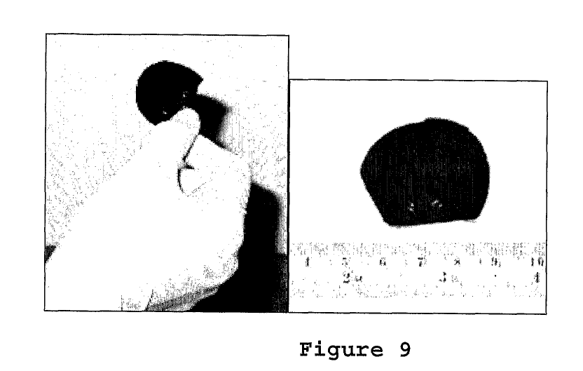

The produced composite was robust and could be readily handled (FIG. 9). Although samples were filtered from a solution, a significant amount of the binder was retained within the carbon nanotube film as determined by TGA (see Table 3 below). The source of the variation in residual weight is not known.

TABLE 3
Summary of TGA results from different samples

| | Binder | Percent of | Residual |
|---|---|---|---|
| Sample | (weight %) | Binder retained | (weight %) |
| DNA | 9 | 10 | 17 |
| Chitosan | 25 | 33 | 11 |
| Triton X-100 | 7 | 0.5 | 17 |

Both the chitosan SWNT and the DNA SWNT films were much stronger than conventional bucky paper samples although the elastic moduli were similar. The mechanical properties do not vary by more than 5% if the DNA or chitosan samples are submerged in water for 5 minutes.

TABLE 4
Physical properties of dry SWNT films

| | Tensile | Elastic | | Specific |
|---|---|---|---|---|
| | Stress | Modulus | Density | Strength |
| Sample | (MPa) | (GPa) | (kg/m³) | (MPa/(Mg/m³) |
| Standard | 16 | 4.0 | 640 | 25 |
| bucky paper | | | | |
| DNA | 76 | 3.3 | 820 | 93 |
| Chitosan | 149 | 3.4 | 920 | 162 |

The conductivity of the chitosan and DNA bound composites were higher than bucky paper, although the electrochemical capacitance was similar or lower (see Table 5 below). The conductivity is more stable for the DNA and chitosan composites relative to standard bucky paper (the conductivity of standard bucky paper decreases over time from about 250 S/cm down to 25 S/cm).

TABLE 5
Electrochemical properties of the SWNT films

| | | | Specific |
|---|---|---|---|
| | Conductivity | Capacitance | Conductivity |
| Sample | (S/cm) | (F/g) | (S · m²/Mg) |
| Standard | 247 | 27 | 3.9 |
| bucky paper | | | |
| DNA | 306 | 27 | 3.7 |
| Chitosan | 290 | 19 | 3.2 |

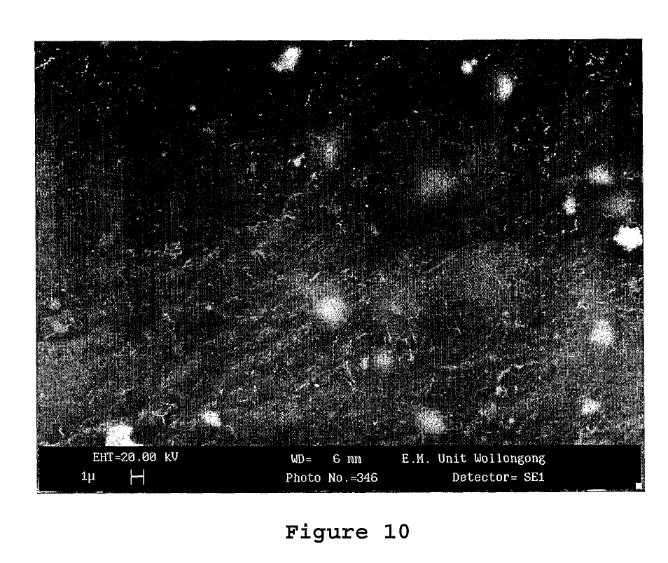

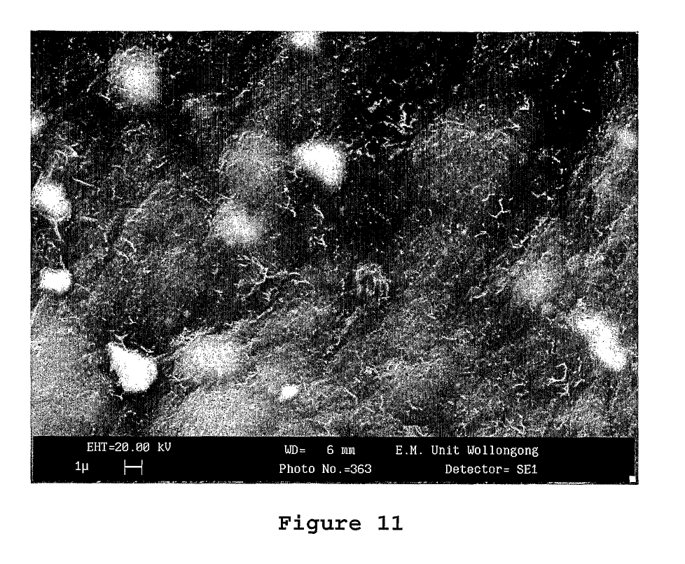

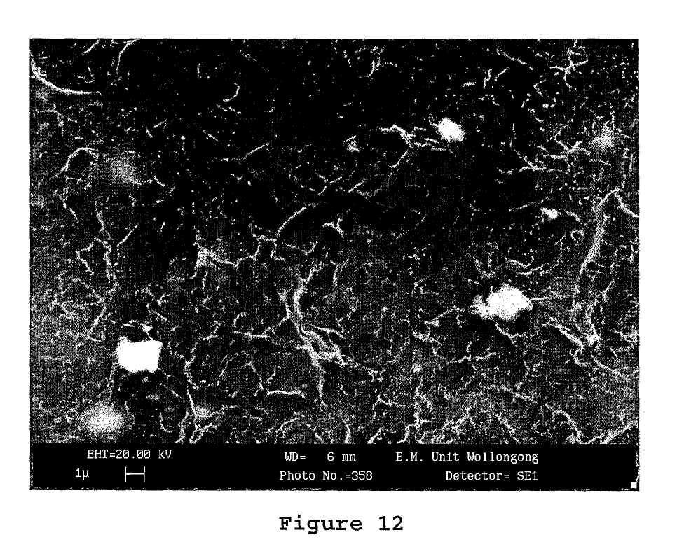

SEM images (see FIGS. 10 to 12) of the filter surface of the CNT assemblies showed that the DNA and Chitosan based films were much rougher than the Triton X film.

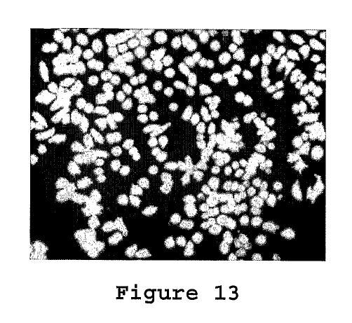

Prolific cell growth was observed on the chitosan—SWNT, and DNA-SWNT composites (FIG. 13). No cell growth was observed on the standard bucky paper sample.

### Discussion

It is believed that the interaction between DNA and SWNT is stronger than the interaction between chitosan and SWNT. Hence, one would expect more DNA to be absorbed onto the SWNT, and be retained in the final composite when compared to chitosan with SWNT. However, TGA analysis shows that there is approximately 6 times the amount of chitosan retained as DNA. It is believed that this is due to the limited solubility of chitosan in water. Indeed, chitosan is insoluble in water at the concentrations reported in this example, and so 2-4 wt % acetic acid was added to make it soluble. It is not understood if the acetic acid concentration within the solution, or filtrate varies during the filtration of the dispersion.

The incorporation of a bio-polymer binder increases the composite density significantly with respect to standard bucky paper, but it is still lower than the 1300-1500 kg/m³ reported for oriented fibres. However, the composite density is still less than that predicted for the carbon nanotube component alone (estimated as 1500 kg/m³), hence the films must be porous. It is believed that the chitosan and DNA compacts the bucky paper as it dries, thereby achieving a higher density.

The elastic modulus of the bio-polymers is similar to that of bucky paper, and much lower than the 9-19 GPa observed in composite fibres. The moderate modulus of the produced composites is evidence that many of the nanotubes are not well dispersed, but rather are retained in large aggregates. A moderate modulus allows the sample to be highly flexible, as the stress concentration is not as high when subjected to bending.

Incorporation of a bio-polymer in the composite resulted in a substantial increase in mechanical strength. The strength of the chitosan-SWNT samples is double that of the freestanding composite films produced by Gheith et al⁵, which were stated as being more than sufficient for soft tissue implants. The strength of the chitosan-SWNT composites is higher than the first generation of oriented polymer-SWNT fibres^{3,6}. It has already been mentioned that the modulus did not increase due to the presence of the bio-polymer, hence the increase in strength is not due to good dispersion of tubes within the binder. The increase in strength is probably due to the binder filling in pores or other defects within the structure that would normally act as stress concentration sites. Preventing nanotube junctions from slipping by the adhesion of a binder is also a possible strengthening mechanism. However, if substantial sliding occurred then one would expect to observe strain of several percent before failure before tube entanglements restrict sliding and promote failure, something that is not observed for standard bucky paper samples or the bio-polymer composites.

For many brittle materials, or brittle composites the observed strength is determined by the size and density of defects (eg. Small cracks). For example, glass fibre exhibits a much higher tensile strength than glass sheet. For bucky paper a major defect with respect to stress concentration would be large pores and the connection between bundles of nanotubes. It is feasible that the bio-polymer would substantially increase the strength by filling in some of the pores, and thereby increasing the strength between clumps.

The specific strength of the three types of samples is at the upper limits of steel or aluminium alloys, but at the lower limit for commercial glass fibre reinforced polymers. The tensile strength of the chitosan-SWNT is better than most common engineering polymers, with only oriented fibres (eg. Nylon or polyethylene) being stronger.

The conductivity of the composites is the most important of the presented results. Composites of DNA-SWNT and chitosan-SWNT exhibited significantly higher conductivity than that of standard bucky paper. It is pertinent to note that the conductivity of bucky paper is substantially lower than that observed for isolated nanotubes. However, it is surprising that the addition of a non-conductive bio-polymer results in an increase of conductivity. Most composites composed of non-conductive binders and carbon nanotubes report conductivities less than 10 S/cm. It was expected that the composite conductivity should be lower on the basis that the non-conductive binder insulates the nanotubes from themselves, and hence limits the number of conductive pathways.

The observed increase in conductivity is most likely due to the poor dispersion. Within the large bundles of nanotubes the local conductivity would be very high, with minimal binder hindering nanotube-nanotube contact. However, electrical contact between bundles is limited by the presence of the insulative binder. Hence, the intra-bundle conductivity would be relatively high, the inter-bundle conductivity relatively low, and the composites conductivity determined by the density of electrical contacts between bundles.

The conductivity is 6 times greater than that reported by Supronowicz et al⁶ which was shown to be sufficient to increase cell proliferation when exposed to an alternating current stimulation. The conductivity was higher than the 167 S/cm reported by Barisci et al³ that was achieved by removing the polymeric binder from the fibre via the process of annealing.

Electrochemical capacitance of macroscopic carbon nanotube samples is difficult to predict. The reported results were similar to bucky paper composed of multiwall nanotubes (12-25 F/g) and SWNT-PVA fibres (7.2 F/g), however they were lower than some polymer-nanotube composites (283 F/g, 180 F/g).

Prolific cell growth on the DNA and chitosan composite samples has been established. It was expected that there would be no cell growth on the standard bucky paper sample, as it contains residual amounts of triton X which is known to disintegrate cells.

Bio-electrodes are one application of the bio-polymer-SWNT composites. The composites exhibit sufficient conductivity, electrochemical capacitance and mechanical properties to be used directly as electrodes implanted into living bodies for the purpose of sensing and stimulation.

Electrodes for biological implants typically consist of platinum or iridium and their derivatives. Here we have produced electrically conducting composites that contain only chitosan and carbon nanotubes, or DNA and carbon nanotubes. Chitosan is known to be biocompatible and is currently used in conjunction with many implants in the human body. Furthermore, functional groups may be added to chitosan to allow further control of the bio-interaction. The bio-compatibility of carbon nanotubes is not known, however initial studies show great promise.

Finally, the filtering process is simple, and allows one to make planar electrodes of almost any dimension. The filtering process can also be used to produce three dimensional shapes including hollow fibres by filtering through a tube.

### Conclusion

Conductive films incorporating non-conductive binders and SWNT have been prepared. The films are much stronger than standard bucky paper, whilst maintaining the conductivity and electrochemistry of bucky paper. One possible application is bio-compatible electrodes.

### REFERENCES

1. M. E. Kozlov, R. C. Capps, W. M. Sampson, V. H. Ebron, J. P. Ferraris, R. H. Baughman, Adv. Mater. 2005, 17, 614.
2. E. Mufloz, D.-S. Suh, S. Collins, M. Selvidge, A. B. Dalton, B. O. Kim, J. M. Razal, G. Ussery, A. O.
Rinzler, M. T. Martinez, R. H. Baughman, Adv. Mater. 2005, 17, 1064.
3. J. N. Barisci, M. Tahhan, G. G. Wallace, S. Badaire, T. Vaugien, M. Maugey, P. Poulin, Adv. Funct. Mater. 2004, 14, 133.
4. Whitten P G, Spinks G M, Wallace G G. Mechanical properties of carbon nanotube paper in ionic liquid and aqueous electrolytes. Carbon 2005; 43(9):1891-96.
5. Gheith M K, Sinani V A, Wicksted J P, Matts R L, Kotov N A. Single-Walled Carbon Nanotube Polyelectrolyte Multilayers and Freestanding Films as a Biocompatible Platform for Neuroprosthetic Implants. Advanced Materials 2005; 17:2663-70.
6. Supronowicz P R, Ajayan P M, Ullmann K R, Arulanandam B P, Metzger D W, Bizios R. Novel current-conducting compositesubstrates for exposing osteoblasts to alternating current stimulation. Journal of Biomedical Materials Research 2001; 59(3):499-506.

It will be understood to persons skilled in the art of the invention that many modifications may be made without departing from the spirit and scope of the invention.

1. A biocompatible composite that is formed into a fibre, mat and/or film structure, comprising nanotubes and at least one biomolecule.

2. A biocompatible composite according to claim 1, wherein the biomolecule is selected from one or more of the group consisting of biological electrolytes, nucleic acids, polyaminoacids, proteins, enzymes, polysaccharides, lipids and/or hormones.

3. A biocompatible composite according to claim 2, wherein the biological electrolyte is selected from one or more of the group consisting of hyaluronic acid (HA), chitosan, heparin, chondroitin sulphate, polyglycol acid (PGA), polylactic acid (PLA), polyamides, poly-2-hydroxy-butyrate (PHB), polycaprolactone (PCL), poly(lactic-co-glycolic) acid (PLGA), protamine sulfate, polyallylamine, polydiallyldimethylammonium, polyethyleneimine, eudragit, gelatin, spermidine, albumin, polyacrylic acid, sodium alginate, polystyrene sulfonate, carrageenin and/or carboxymethylcellulose.

4. A biocomptabile composite according to claim 2, wherein the nucleic acid is selected from one or more of the group consisting of DNA, cDNA, RNA, oligonucleotide, oligoribonucleotide, modified oligonucleotide, modified oligoribonucleotide and/or peptide nucleic acid (PNA) or hybrid molecules thereof.

5. A biocompatible composite according to claim 2, wherein the polyamino acid is selected from one or more of the group consisting of poly-L-lysine, poly-L-arginine, poly-L-aspartic acid, poly-D-glutamaic acid, poly-L-glutamaic acid, poly-L-histidine and/or poly-(DL)-lactide.

6. (canceled)

7. (canceled)

8. (canceled)

9. (canceled)

10. (canceled)

11. A biocompatible composite according to claim 1, wherein the biomolecule is selected from one or more of the group consisting of hyaluronic acid (HA), chitosan, heparin, chondroitin sulphate, DNA and/or poly-L-lysine.

12. A biocompatible composite according to claim 1, wherein the nanotubes are selected from one or more of the group consisting of carbon nanotubes, metal oxide nanotubes and/or peptidyl nanotubes.

13. (canceled)

14. A biocompatible composite according to claim 1, wherein the nanotubes are SWNTs and/or MWNTs.

15. A biocompatible composite according to claim 1, wherein the composite is a fibre selected from the group consisting of DNA-SWNT-chitosan fibres, HA-SWNT-chitosan fibres, chitosan-SWNT-chondroitin sulphate fibres and chitosan-SWNT-heparin fibres or a film selected from the group consisting of chitosan-SWNT-films, DNA-SWNT films and poly-1-lysine-SWNT films.

16. A biocompatible compatible composite according to claim 1, wherein said biomolecule is present in an amount in the range of 10-50% based on the total weight of the composite.

17. A biocompatible composite according to claim 1, wherein said composite further comprises an additive in an amount in the range of 1 to 50% based on the total weight of the composite, wherein said additive is selected from the group consisting of drugs, growth factors, hormones, antibiotics, mRNA, DNA, steroids, antibodies and/or radio-isotopes.

18. The biocompatible composite according to claim 1, wherein the composite has a tensile stress in the range of 50-200 MPa.

19. The biocompatible composite according to claim 1, wherein the composite has an elastic modulus in the range of 1-20 GPa.

20. The biocompatible composite according to claim 1, wherein the composite has a density in the range of 0.6-1 g/m3.

21. The biocompatible composite according to claim 1, wherein the composite has an electrical conductivity is in the range of 0.5 to 400 S/cm.

22. A process for preparing a biocompatible composite which comprises the steps of:
(i) forming a dispersing media comprising nanotubes and at least one biomolecule; and either
(ii) introducing the dispersing media of step (i) into a coagulating media optionally comprising at least one biomolecule so as to form a continuous fibre; or
(iii) filtering the dispersing media of step (i).

23. A process according to claim 22, wherein at least one biomolecule is present in both the dispersing media of step (i) and the coagulating media of step (ii).

24. A process according to claim 23 wherein the biomolecule in the dispersing media possesses a charge opposite to the charge in the biomolecule of the coagulating media.

25. A process for preparing a biocompatible composite which comprises the steps of:
(i) forming a dispersing media comprising nanotubes; and
(ii) introducing the dispersing media of step (i) into a coagulating media comprising at least one biomolecule so as to form a continuous fibre.

26. A process according to claim 22, wherein the dispersing media and/or coagulating media is a solution with a viscosity up to about 200 cps.

27. (canceled)

28. A process according to claim 22, wherein the dispersing media is formed by sonication.

29. A process according to claim 22, wherein the nanotube concentration in the dispersing media is in the range of 0.2 to 0.5 wt % based on the total weight of the dispersing media.

30. A process according to claim 22, wherein the ratio of biomolecule to nanotubes is in the range of 1:1 to 5:1.

31. A process according to claim 22, wherein coagulation involves spinning the nanotube or biomolecule-nanotube dispersion into the coagulating media.

32. A process according to claim 31, wherein the nanotube or biomolecule-nanotube dispersion is spun into the coagulating media by injecting the dispersion through an orifice into the spinning coagulating media.

33. A process according to claim 32, wherein the injection occurs at a rate in the range of 150 to 300 ml/hr.

34. A process according to claim 32, wherein the spinning coagulating media spins at a rate in the range of 25 to 60 rpm.

35. A biocompatible composite prepared by a process according to claim 22.

36. A medical device composed wholly or partly of the composite according to claim 1.

37. A medical device according to claim 36, wherein the medical device is a bio-electrode, bio-fuel cell, or substrate for electrically stimulated bio-growth.

38. A medical device according to claim 36, wherein the medical device is used in pacemaker electrodes, ECG pads, biosensors, muscle stimulation, epilepsy control, or electrical stimulated cell regrowth.

39. A process according to claim 25, wherein the dispersing media and/or coagulating media is a solution with a viscosity up to about 200 cps.

40. A process according to claim 25, wherein the dispersing media is formed by sonication.

41. A process according to claim 25, wherein the nanotube concentration in the dispersing media is in the range of 0.2 to 0.5 wt % based on the total weight of the dispersing media.

42. A process according to claim 25, wherein the ratio of biomolecule to nanotubes is in the range of 1:1 to 5:1.

43. A process according to claim 25, wherein coagulation involves spinning the nanotube or biomolecule-nanotube dispersion into the coagulating media.

44. A process according to claim 43, wherein the nanotube or biomolecule-nanotube dispersion is spun into the coagulating media by injecting the dispersion through an orifice into the spinning coagulating media.

45. A process according to claim 44, wherein the injection occurs at a rate in the range of 150 to 300 ml/hr.

46. A process according to claim 44, wherein the spinning coagulating media spins at a rate in the range of 25 to 60 rpm.

47. A biocompatible composite prepared by a process according to claim 25.

48. A medical device composed wholly or partly of the composite according to claim 22.

49. A medical device composed wholly or partly of the composite according to claim 25.

50. A medical device according to claim 48, wherein the medical device is a bio-electrode, bio-fuel cell, or substrate for electrically stimulated bio-growth.

51. A medical device according to claim 49, wherein the medical device is a bio-electrode, bio-fuel cell, or substrate for electrically stimulated bio-growth.

52. A medical device according to claim 48, wherein the medical device is used in pacemaker electrodes, ECG pads, biosensors, muscle stimulation, epilepsy control, or electrical stimulated cell regrowth.

53. A medical device according to claim 49, wherein the medical device is used in pacemaker electrodes, ECG pads, biosensors, muscle stimulation, epilepsy control, or electrical stimulated cell regrowth.
